(19) United States
(12) Patent Application Publication
(10) Pub. No.: US 2018/0071428 A1
(43) Pub. Date: Mar. 15, 2018

(54) WOUND DRESSING

(71) Applicant: Sefar AG, Heiden (CH)

(72) Inventors: Gerd GERDES, Abtwil (CH); Jérémie WEBER, Klaus (AT)

(73) Assignee: Sefar AG, Heiden (CH)

(21) Appl. No.: 15/560,357

(22) PCT Filed: Oct. 24, 2016
(86) PCT No.: PCT/EP2016/075495

§ 371 Date: Sep. 21, 2017

(30) Foreign Application Priority Data
Apr. 14, 2016 (EP) 16165403.3

(51) Int. Cl. A61L 15/56; A61F 13/00; A61L 15/44

(52) U.S. Cl. CPC A61L 15/56; A61F 13/00063; A61L 15/58; A61F 13/00059; A61F 2013/00927; A61L 15/44

(57) ABSTRACT

The invention relates to a wound dressing and a method for producing a wound dressing with a marker material for indicating a condition of a wound, in particular a microbiological wound milieus. The invention is characterized in that the wound dressing has a contact side which is formed by a biocompatible monofilament fabric of monofilament threads with a smooth surface and in that on the monofilament fabric on its upper side facing away from the wound at least one cover element is fixed which is provided with the marker material.

The invention relates to a wound dressing with a marker material for indicating a condition of a wound, in particular a microbiological wound milieus, in accordance with the preamble of claim 1.

The invention further relates to a method for producing a wound dressing with a marker material in accordance with claim 15.

From WO 2000/024438 A1 a composition is known that can indicate a microbiological contamination on medical devices and materials. This indicator comprises a substance or a substance composition which, depending on the presence of a microbiological environment, can cause a color change. For medical personnel this is a sign that a contamination is present or has at least reached a certain level.

Furthermore, it is known that such indicator substances are also provided for wound dressings. Through this, medical personnel can get an indication of a wound development at an early stage. In particular, by way of a corresponding indicator notice can be given that a change of the wound dressing is advisable or necessary since a certain level of contamination has been reached.

However, the fluctuations in the response of the indicator material are not inconsiderable. One reason for this can be seen in the fact that the leaking of exudate or secretions from the wound can have an influence not inconsiderable on the point in time when a color change is indicated. If the wound dressing material is in direct contact with the wound an early indication can take place. If, however, the wound dressing is positioned at a certain distance to the tissue of the wound the indication of a contamination may perhaps occur with a delay. This can be detrimental to the wound healing process.

The invention is based on the object to provide a wound dressing and a method for producing the wound dressing, with which a condition of the wound can be indicated in a particularly reliable manner.

The object is achieved on the one hand by a wound dressing having the features of claim 1 and on the other hand by a method having the features of claim 15. Preferred embodiments are stated in the dependent claims.

The invention is characterized in that the wound dressing has a contact side which is formed by a biocompatible monofilament fabric of monofilament threads with a smooth surface and in that on the monofilament fabric on its upper side facing away from the wound at least one cover element is fixed which is provided with the marker material.

One aspect of the invention resides in the fact that the cover element with the marker material is not in direct contact with the injured tissue of the wound. In fact, between the wound and the cover element with the marker material a separating layer is provided which, according to the invention, is formed of a biocompatible monofilament fabric. The monofilament fabric is formed of fine wire-like monofilament yarns. Compared to staple fibers or multifilament yarns a penetration of wound secretions or marker material into the monofilament yarns themselves and a thereby resultant increased contamination is structurally not possible in the case of monofilament yarns. In this way, a premature indication of a not yet critical condition of contamination can be prevented. At the same time the cover element with the marker material can be designed such that it only responds when a specific contamination is present behind the monofilament fabric as a separating layer.

Moreover, through the separating layer of the monofilament fabric an irritation of the wound by the cover element and the marker material provided therein is counteracted. Such interactions can lead to a disorder of wound healing.

According to a further aspect of the invention the monofilament fabric is of biocompatible design. The choice of the raw materials and the processing into the monofilament fabric are devised such that the monofilament fabric is non-irritating, in particular non-cytotoxic, pyrogen-free and hypoallergenic. Fabrics, as they are usually employed in the industry, are generally not biocompatible. However, biocompatible fabrics are known from the field of blood filtration. Biocompatibility of the fabric can be assumed in particular if the requirements according to the relevant chapter of ISO 10993 or of the USP Plastics Class VI Test (United States Pharmacopeia) are met.

Through biocompatibility and the structure of the yarn it is ensured that on the one hand an irritation of the injured tissue material by the wound dressing material is largely prevented and on the other hand the risk of the wound being harmed, in particular being torn open during removal of the wound dressing material is counteracted.

Moreover, the biocompatible monofilament fabric can be of such stable design that it serves as a carrier layer, on which at least one cover element is firmly fixed. This cover element can thereby itself have a single layer or several layers. According to the purpose of application of the wound dressing the cover element can additionally be designed in a desired way, e.g. to receive wound secretions or to apply means promoting wound healing such as an ointment or a liquid.

According to the invention different elements and materials can be provided to form the cover element as these are arranged on the upper side facing away from the contact side of the monofilament fabric and therefore do not make direct contact with the tissue material in the wound area. The cover element itself can have a multi-layer structure.

A preferred embodiment of the invention resides in the fact that the monofilament fabric is designed as a single-weave fabric with an open-mesh structure having a pore size ranging from 5 μm to 500 μm, preferably from 20 μm to 300 μm, and that the pores constitute a proportion of 15% to 70% of the surface of the monofilament fabric. Thus, according to this embodiment the monofilament fabric according to the invention is particularly fine-pored, allowing on the one hand air and liquid to pass through the pores while on the other hand a coadunate or clutching of the monofilament fabric by tissue material is hardly feasible. In this way, a protective and yet permeable wound dressing is created which however, still ensures a clear demarcation and therefore easy removability from the wound. Good permeability for air and liquid is given in particular by the fact that the pores constitute a proportion of 15% to 70%, preferably 40% to 70% of the surface of the monofilament fabric. In this way, a high permeability is attained.

Thereby, the monofilament fabric is designed as a single-weave fabric having a substantially two-dimensional fabric construction with a single warp thread system and a single weft thread system. Moreover, by making use of a transparent thread material for the monofilament threads in combination with the large pore surface a very high transparency is achieved. Despite a relatively thin fabric thickness a sufficient stability of the carrier layer formed by the monofilament fabric, along with a good fabric openness, can be achieved by a plain weave which is a taffeta 1:1. Another stable construction can be achieved by a twill weave in any pattern repeats, preferably in a 1:2, 1:3, 2:2 and 3:3 weave regarding warp thread/weft thread arrangement. Another stable weave is possible by a satin weave in any pattern repeats, by particular preference in a 1:7 satin weave regarding warp thread/weft thread arrangement. A further possible stable weave for the monofilament fabric is a plain Dutch weave.

According to a further development of the invention it is preferred that the monofilament threads of the monofilament fabric are formed of PA, PET, PP, PVDF and/or PTFE and have a diameter size ranging between 20 μm and 500 μm, preferably between 30 μm and 150 μm. The monofilament threads thereby are produced of a particularly pure basic material so that these comply with food legislation. Pyrogen-free and non-cytotoxic fabrics can be produced. A particularly preferred material is polyester in a purified and biocompatible state. Depending on the case of application the thread diameters can vary but preferably they are kept as small as possible. In particular, the monofilament threads are designed with a longitudinal rib structure, in which the longitudinal ribs protrude radially from a core diameter of the monofilament thread by a maximum of 0.5 μm to 5 μm. This very fine rib design furthermore ensures a relatively smooth surface of the monofilament threads while providing at the same time adequate stability at the points of intersection of the monofilament threads in the fabric and therefore a stable shape of the pores. In addition to the usual finishing comprising a thermofixing the monofilament fabric can be calendered.

Another preferred embodiment of the invention resides in the fact that the contact side is provided at least in some areas with an adhesive layer. Usually, the adhesive layer is an application of adhesive that is suitable for being applied to the skin. The adhesive layer can be applied directly onto the contact side of the monofilament fabric.

Preferably, this embodiment of the invention is developed further in that the adhesive layer is applied in a strip-shaped manner along a marginal area of the contact side of the monofilament fabric. In a further development the upper side of the marginal area of the monofilament fabric which faces away from the contact side can be kept free from the cover element.

Hence, in this embodiment the cover element does not extend over the entire surface of the monofilament fabric but only in a center area while a strip-shaped marginal area is kept free. The marginal area can only extend along a partial area of the external circumference or be designed along the entire external circumference and therefore be closed in a ring-shaped manner. The strip-shaped adhesive layer allows the wound dressing material to be fixed in a spaced manner from the wound. By keeping the upper side of the marginal area free a sufficient access of air is rendered possible and an irritation of the skin caused by the adhesive layer is counteracted.

The marginal area of the wound dressing material can be trimmed, in particular through ultrasonic trimming or thermal trimming (heat cutting), in which the cut edges are welded and rounded off. The adhesive layer can also be applied to a contact element which is in turn applied along the marginal area of the monofilament fabric. This contact element thereby can be folded around the marginal edge of the monfilament fabric and can trim this in addition so as to thereby increase the wearing comfort further.

According to a further development of the invention it is advantageous for the cover element to have a fabric, a non-woven textile, a film, a membrane and/or a foam. In particular, the cover element itself can have a layer-wise or multi-ply structure, in which the individual layers or plies are themselves formed of identical or different materials. When using a fabric, a multifilament fabric with an increased absorptive capacity can also in particular be applied in addition to a further monofilament fabric. For this purpose, non-woven textiles, such as a knitted fabric, a fleece, felt or the like or even cotton wool can also be employed. These materials are also well-suited to receive care substances or medically effective substances. To protect against the entry of liquids, gases or other physical influences from the outside provision can be made for a film, a membrane and/or a foam. By preference, the membrane thereby can be semi-permeable, allowing an exchange of gas while preventing the entry of liquid from the outside into the wound or, vice versa, the leakage of liquid from the wound into an external area.

According to the invention provision is made for the cover element to be spot-welded to the monofilament fabric. This can be implemented through thermal welding or preferably through ultrasonic welding. The welding process can also be effected during heat cutting or laser cutting in the marginal area. Along the marginal area the cover element can be welded in a linear manner to the monofilament fabric so that the cover element is in particular closed in an all-embracing or annular manner on its external side.

Another advantageous embodiment of the invention resides in the fact that the cover element has a transparent external cover layer. This can be connected to the monofilament fabric by forming at least one receiving area. The transparent cover layer protects the wound dressing towards the outside. Depending on the application the cover layer can be permeable or be a barrier for humidity and/or gas. The at least one receiving area thereby can be a hollow space, in which a further element is provided to receive liquid or a substance. The external cover layer thereby can be a film or a membrane in particular which forms a liquid-tight closure towards the outside. In this way, it can be ensured in particular that items of clothing worn on the wound dressing material do not become soiled by substances from the wound dressing material or by wound secretions.

To reduce tissue irritation provision is made according to a further embodiment variant of the invention that the thickness of the upper side formed by the cover element amounts to 60% to 98% of the overall thickness of the wound dressing material. The biocompatible monofilament fabric is very fine and has a thickness of 40 μm to 1 mm, preferably ranging between 70 μm and 300 μm. Depending on the case of application the cover element which forms the upper side of the wound dressing material can range from 60 μm up to a few milimeters.

To promote wound healing provision is made according to a further embodiment pursuant to the invention that several cover elements are provided, wherein at least one further cover element has a care substance or medically effective substance or is designed to receive wound secretions. The cover element can be a reservoir with a liquid or gel filling or be designed to receive powders or solids.

Basically, provision is made for the monofilament threads to be produced of a plastic material high in purity and in such a manner that these have a surface as smooth as possible by themselves. An advantageous embodiment of the invention resides in the fact that the monofilament fabric or the monofilament threads that form the monofilament fabric are provided with a coating. With this coating the surface can be treated and influenced in a desired way. In particular, a hydrophilic or hydrophobic coating is possible. Moreover, the coarseness and adhering property of the surface can be reduced further. In addition to such a surface coating with suitable materials it is also possible to adjust a different surface modification, especially a smoothing of the thread surfaces or the surface of the finished monofilament fabric.

Another advantageous embodiment of the invention can be seen in the fact that the monofilament fabric is woven with a uniform defined pore size, wherein the pore sizes do not differ from each other by more than 10%, preferably by less than 5%. Hence, a monofilament fabric is created that has a highly uniform pore size across the entire surface. Depending on the purpose of application a pore size as appropriate as possible can thus be provided which allows on the one hand e.g. the passage of air or liquid in the desired way while on the other hand the risk of ingrowth or clutching of tissue material is counteracted to a large degree.

Another preferred embodiment of the invention resides in the fact that at least one cover element is imprinted or coated with the marker material. Application thereby can be made on one or on both sides. The imprinting or coating can exclusively be carried out as a surface treatment so that the consumption of marker material is limited. Imprinting or coating proves to be especially advantageous if the cover element is produced of a web material.

Basically, as marker material, which serves as an indicator for indicating the condition of the wound, in particular a microbiological wound milieus and more particularly a contamination, any suitable marker material can be used. According to a further development of the invention it is especially preferred that the marker material comprises at least one colorant, in particular a reactive colorant, a fluorescent substance, a pH-sensitive substance, enzymes, cells, a hydrogel, a termperature-sensitive substance, a salt, an ointment and/or alginates.

According to the invention provision is made for a method for producing a wound dressing with a marker material, in particular as described beforehand, in which a biocompatible monofilament fabric is formed as a first web material, a cover element can be formed as a second web material that is provided with the marker material, the first web material and the second web material are joined, wherein the biocompatible monofilament fabric and the cover element are firmly connected, in particular welded to each other, and the wound dressing material is produced as a tape. The tape thus produced can be wound up to a roll or cut, tailored and imprinted or coated with the marker material in the desired way.

The method according to the invention permits an efficient production of the wound dressing in a continuous process. The basic materials are preferably supplied as tape-shaped webs from rolls. The cover element thereby can be a prefabricated web material that consists of several plies. It is also possible that the cover element is formed of several web materials that are supplied simultaneously and connected to the monofilament fabric.

1. Wound dressing with a marker material for indicating a condition of a wound, in particular a microbiological wound milieus,
wherein
the wound dressing has a contact side which is formed by a biocompatible monofilament fabric of monofilament threads with a smooth surface and
on the monofilament fabric on its upper side facing away from the wound at least one cover element is fixed which is provided with the marker material.

2. Wound dressing according to claim 1,
wherein
the monofilament fabric is formed as a single-weave fabric with an open-mesh structure having a pore size ranging from 5 μm to 500 μm, preferably from 20 μm to 300 μm, and
the pores constitute a proportion of 15% to 70% of the surface of the monofilament fabric.

3. Wound dressing according to claim 1,
wherein
the monofilament threads of the monofilament fabric are formed of PA, PET, PP, PVDF and/or PTFE and have a diameter size ranging between 20 μm and 500 μm, preferably between 30 μm and 150 μm.

4. Wound dressing according to claim 1,
wherein
the contact side is provided at least in some areas with an adhesive layer.

5. Wound dressing according to claim 4,
wherein
the adhesive layer is applied in a strip-shaped manner along a marginal area of the contact side of the monofilament fabric.

6. Wound dressing according to claim 1,
wherein
the cover element has a fabric, a non-woven textile, a film, a membrane and/or a foam.

7. Wound dressing according to claim 1,
wherein
the cover element is spot-welded to the monofilament fabric, in particular through ultrasonic welding.

8. Wound dressing according to claim 1,
wherein
the cover element has a transparent external protective layer.

9. Wound dressing according to claim 1,
wherein
the thickness of the upper side formed by the cover element amounts to 60% to 98% of the overall thickness of the wound dressing material.

10. Wound dressing according to claim 1,
wherein
several cover elements are provided, wherein at least one further cover element has a care substance or medically effective substance or is designed to receive wound secretions.

11. Wound dressing according to claim 1,
wherein
the monofilament fabric or the monofilament threads that form the monofilament fabric are provided with a coating.

12. Wound dressing according to claim 1,
wherein
the monofilament fabric is woven with a uniform defined pore size, wherein the pore sizes do not differ from each other by more than 10%.

13. Wound dressing according to claim 1,
wherein
the at least one cover element is imprinted or coated with the marker material.

14. Wound dressing according to claim 1,
wherein
the marker material comprises at least one colorant, in particular a reactive colorant, a fluorescent substance, a pH-sensitive substance, enzymes, cells, a hydrogel, a temperature-sensitive substance, a salt, an ointment and/or alginates.

15. Method for producing a wound dressing with a marker material according to claim 1, in which
a biocompatible monofilament fabric is formed as a first web material,
a cover element is formed as a second web material, and
the first web material and the second web material are joined, wherein the biocompatible monofilament fabric and the cover element are firmly connected to each other through spot welding,
wherein the cover element is provided with the marker material.

16. Method according to claim 15,
wherein
the marker material is applied to the cover element, in particular imprinted or coated thereon, before and/or after joining of the two web materials.
